(19) United States
(12) Patent Application Publication
(10) Pub. No.: US 2014/0039554 A1
(43) Pub. Date: Feb. 6, 2014

(54) APPARATUS FOR FIXING A CERVICAL SPINE HAVING SELF TENSION PART

(71) Applicants: Seo-Kon Kim, Gyeonggi-do (KR); II Kim, Seoul (KR); Do-Heum Yoon, Seoul (KR)

(72) Inventors: Seo-Kon Kim, Gyeonggi-do (KR); II Kim, Seoul (KR); Do-Heum Yoon, Seoul (KR)

(21) Appl. No.: 13/955,418

(22) Filed: Jul. 31, 2013

(30) Foreign Application Priority Data
Aug. 3, 2012 (KR) 10-2012-85115
Jul. 24, 2013 (KR) 10-2013-87576

(51) Int. Cl. A61B 17/70

(52) U.S. Cl. CPC A61B 17/7059
USPC 606/246

(57) ABSTRACT

An apparatus for fixing a cervical spine having a self tension part used in orthopedic surgery or neurosurgery when executing an anterior fixation operation on cervical spines is disclosed, including: an extension groove formed through a plate between the insertion holes and the center of the plate; a channel groove formed with stepped surfaces at both sides at the boundary of the insertion holes and the extension groove; a guide that is connected to the channel groove, rotates left/rightward in the channel groove and locks the screw to be inserted into the insertion holes; and a tension part that gives a pushing force the guide to be pushed toward the center of the plate and allows the guide to be rotated in the channel groove.

### RELATED APPLICATION

This application claims the benefit of Korean Patent Application No. 10-2012-85115, filed on Aug. 3, 2012 and Korean Patent Application No. 10-2013-87576, filed on Jul. 24, 2013, the disclosure of which is entirely incorporated herein by reference.

### TECHNICAL FIELD

The present disclosure relates to an apparatus for fixing a cervical spine having a self tension part, which is used for fixing a front of cervical spine in orthopedic surgery or neurosurgery, and more specifically, to an apparatus for fixing a cervical spine having a self tension part, which includes; an extension groove formed through a guide plate between an insertion hole and a center of the plate wherein a screw is inserted into one or more insertion holes that are formed through the plate; a tension part formed inside of the extension groove and giving force for pushing the guide toward the center of the guide plate; a channel groove provided at the boundary of the insertion holes and the extension groove and formed with stepped surfaces at both sides; and a guide that is connected to the channel groove and rotates left/rightward in the channel groove, and locks the screw to be inserted into the insertion holes while rotating front/rearward with the pushing force of the tension part.

### BACKGROUND ART

Generally, when treating cervical disc diseases, cervical disc, cervical syndrome, cervical fractures, etc. in orthopedic surgery or neurosurgery, each vertebral body forming cervical vertebrae has to be fixed not to move about, thus for the fixation, a apparatus for fixing a cervical spine which includes a plate of a flat shape and a screw for fixing the plate to the cervical vertebrae are used. fixing a cervical spine briefly, the affected part is incised at the anterior of a neck portion of a patient and cervical vertebrae is exposed, and then cervical disc of the affected part is removed and surgical object of a cage, etc. is inserted between the vertebral bodies from which disc is removed, and then the plate is positioned in a top vertebral body and a bottom vertebral body of the portion into which the cage is inserted and the plate is fixed with a screw.

The apparatus for fixing a cervical spine, as operated above, will firmly fix the cage that is inserted between vertebral bodies with the fixing force of the screw and the sustaining force of the plate.

However, the screw fixed to vertebral bodies may be unscrewed due to the activity of the patient and retreat from the plate backward, causing a vibration of the cage and deterioration of the patient's recovery.

Therefore, in order to prevent phenomena in which the screw of the apparatus for fixing a cervical spine is unscrewed and retreats, “Neck Portion Plate System”(Disclosed Technology 1) has been disclosed in Korean Patent Laid-Open No. 10-2009-86387 and “Apparatus for Fixing Cervical Spine”(Disclosed Technology 2) has been disclosed in Korean Patent Laid-Open No. 10-2005-11167, as conventional arts.

Disclosed Technology 1 is configured to include a rotation element that is installed to be inserted into a hole of a screw on a plate of a cervical plate system, and Disclosed Technology 2 is configured to include a head fixing member to be hooked over the head of a fixing screw, between insertion holes of fixation screws formed on the plate.

However, in technologies for fixing a cervical spine according to the conventional arts, the rotation element (Disclosed Technology 1) for preventing retreatment of the screw and the head fixing member (Disclosed Technology 2) have to be used essentially, thereby increasing the number of components, making the structure and the operation thereof to be complicated, and thus manufacturing cost of the apparatus for fixing a cervical spine will increase and there may be a possibility of erroneous operation of the apparatus.

The invention disclosed in this background of the invention section is only for enhancement of understanding of the background of the invention and should not be taken as an acknowledgement or any form of suggestion that this information forms the prior art already known to a person skilled in the art.

### SUMMARY OF THE INVENTION

The present invention has been made in an effort to solve the above-described problems associated with prior art and it is an object of the present invention to provide an apparatus for fixing a cervical spine in which a retreatment phenomenon of a screw can be prevented by firmly fixing the screw that is inserted at various angles to a plate at an operation by using, and further the operation with the apparatus for fixing a cervical can be performed easily

In order to achieve the object of the present invention, the present invention provides an apparatus for fixing a cervical spine having a self tension part, including: an extension groove formed through a plate between the insertion holes and the center of the plate; a channel groove formed with stepped surfaces at both sides at the boundary of the insertion holes and the extension groove; a guide that is connected to the channel groove, rotates left/rightward in the channel groove and locks the screw to be inserted into the insertion holes; and a tension part that gives a pushing force the guide to be pushed toward the center of the plate and allows the guide to be rotated in the channel groove.

### BRIEF DESCRIPTION OF DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 is a perspective view showing an apparatus for fixing a cervical spine according to a first exemplary embodiment of the present invention;

FIG. 2 is a plan view showing an apparatus for fixing a cervical spine according to a first exemplary embodiment of the present invention;

FIG. 3 is an exploded perspective view showing an apparatus for fixing a cervical spine according to a first exemplary embodiment of the present invention;

FIGS. 4 to 5 are views showing operating conditions of the guides of an apparatus for fixing a cervical spine according to a first exemplary embodiment of the present invention;

FIG. 6 is a view showing an operating condition of an apparatus for fixing a cervical spine inserted with a screw according to a first exemplary embodiment of the present invention;

FIG. 7 is a perspective view showing an apparatus for fixing a cervical spine inserted with a screw according to a first exemplary embodiment of the present invention;

FIG. 8 is a perspective view showing an apparatus for fixing a cervical spine according to a second exemplary embodiment of the present invention;

FIG. 9 is a plan view showing an apparatus for fixing a cervical spine according to a second exemplary embodiment of the present invention;

FIG. 10 is a bottom view showing an apparatus for fixing a cervical spine according to a second exemplary embodiment of the present invention;

FIG. 11 is an exploded perspective view showing an apparatus for fixing a cervical spine according to a second exemplary embodiment of the present invention;

FIG. 12 is a sectional view taken along line A-A of FIG. 9;

FIG. 13 is a view showing an operating condition of a guide of an apparatus for fixing a cervical spine according to a second exemplary embodiment of the present invention; and

FIG. 14 is a view showing an operating condition of an apparatus for fixing a cervical spine inserted with a screw according to a second exemplary embodiment of the present invention.

### DETAILED DESCRIPTION

Hereinafter, an apparatus for fixing a cervical spine having a self tension part according to an exemplary embodiment of the present invention will be described, referring to the accompanying drawings.

However, the disclosed drawings are provided exemplarily for transferring the spirit of the present invention to a skilled ordinary person. Accordingly, the present invention is not limited to the drawings referred below and may be implemented with other aspect.

Additionally, the terms used in the present invention, unless defined as other meanings, have ordinary meanings such that a person who has an ordinary skill in the art to which the present invention pertains can understand them, and the detailed description of a publicly known function and constitution that may obscure a gist of the present invention will be omitted in the following.

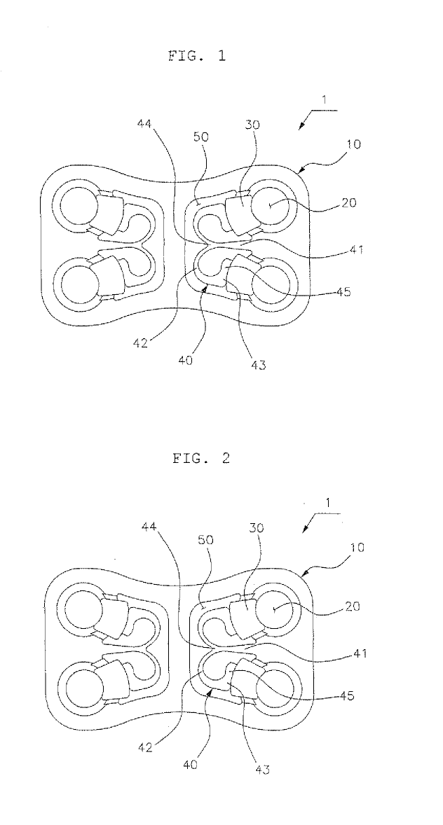

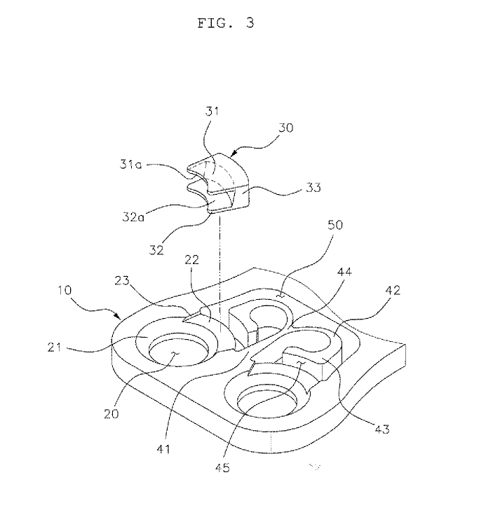

FIG. 1 is a perspective view showing an apparatus for fixing a cervical spine according to a first exemplary embodiment of the present invention, and FIG. 2 is a plan view showing an apparatus for fixing a cervical spine according to a first exemplary embodiment of the present invention, and FIG. 3 is an exploded perspective view showing an apparatus for fixing a cervical spine according to a first exemplary embodiment of the present invention.

Referring to the drawings, the apparatus for fixing a cervical spine 1 according to a first exemplary embodiment of the present invention is provided with a plate 10 of a flat shape.

The plate 10 may be made preferably of a rigid metal such as Titanium, etc.

The plate 10 is shaped as a quadrangle wherein each edge thereof is curved. Further, inside of each edge of the plate 10, one or more insertion holes 20 into which screws 100 for fixing the plate 10 to the cervical spine are inserted are perforated through the plate 10.

In the first exemplary embodiment shown in the drawings, four insertion holes 20 are provided, however the number of the insertion hole is not limited thereto, and thus two, six, eight or more insertion holes may be provided depending on the fixation level of a cervical spine fixing operation.

Further, an inner wall 21 of the insertion hole 20 is curved to be meshed with the shape of a head 101 of a screw 100, which will be described later.

Additionally, an extension groove 50 is provided through the plate 10 between the insertion holes 20 and the center of the plate 10, and at the boundary of the insertion holes 20 and the extension groove 50, a channel groove 22 is formed with stepped surfaces 23 each having a predetermined height from the surface of the plate 10 at both sides.

A guide 30 to be described later is inserted into the channel groove 22, and the inserted guide 30 may be rotated right/leftward in the channel groove 22 while following the entering direction of the screw 100.

The guide 30 is shaped as an alligator clip wherein it is formed integrally by connecting an upper jaw 31 and a lower jaw 32 in flat shapes, which are extended horizontally, with a connective surface 33 that is extended vertically.

The guide 30 as described above is connected to the plate such that the channel groove 22 formed in the plate 10 is inserted between the bottom surface 31 a of the upper jaw 31 and the upper surface 32a of the lower jaw 32 of the guide.

Then, the upper jaw 31 of the connected guide 30 passes the upper surface of the channel groove 22 and protrudes toward the center of the insertion holes 20, and the bottom surface 31a of the protruded upper jaw 31 contacts an upper surface of the head 101 of the screw 100 that is entering into the insertion hole 20, thereby locking the screw 100 and providing a hooking operation to the screw 100 not to retreat.

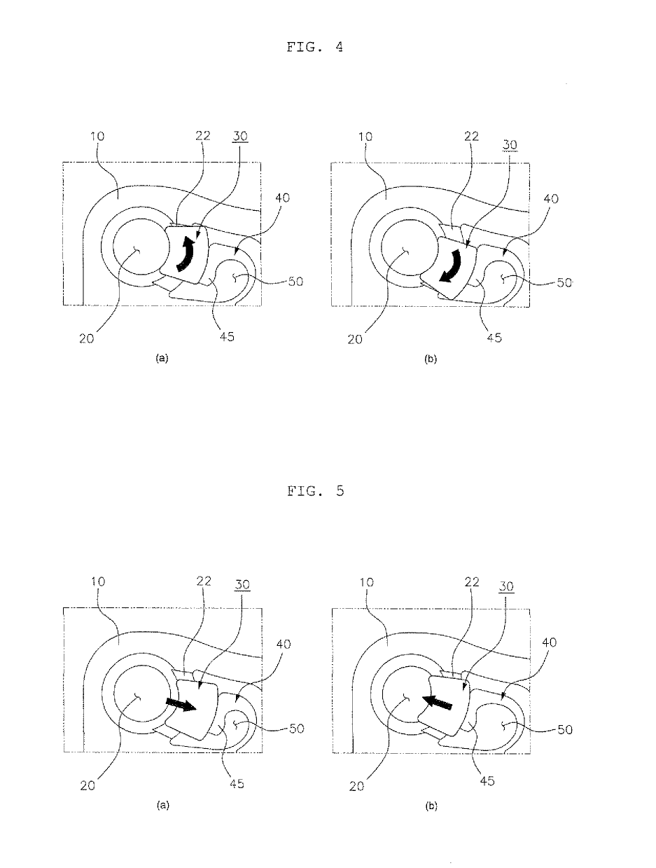

As shown in FIG. 4, the engaged guide 30 can rotate right/leftward until the upper jaw 31 and the lower jaw 32 are hooked over the stepped surfaces 23 at both sides of the channel groove 22.

Additionally, a tension part 40 for pushing the guide 30 toward the center of the plate 10 is formed inside the extension groove 50.

As shown in FIGS. 2 to 3, the tension part 40 includes: a base 41 extending toward the center of the plate 10 at one side of an edge of the extension groove 50; a first curved part 42 that is bent towards the guide 30 at the end of the base 41; and a second curved part 43 that is bent in parallel with the connective surface 33 of the guide 30 at the end of the first curved part 42 wherein the contact surface between the second curved part 43 and the connective surface of the guide 30 forms an arm 45.

At this time, another tension part 40 may be formed additionally for an another insertion hole 20 from the end of the base 41 of the tension part 40 wherein a branching part 44 is formed at the point where the another tension part starts to be formed.

The arm 45 of the tension part 40 will have a self-tension force for pushing the guide 30 to be in contact with the arm 45, since the elasticity from the bending shapes of the first curved part 42 and the second curved part 43 is applied to the arm 45.

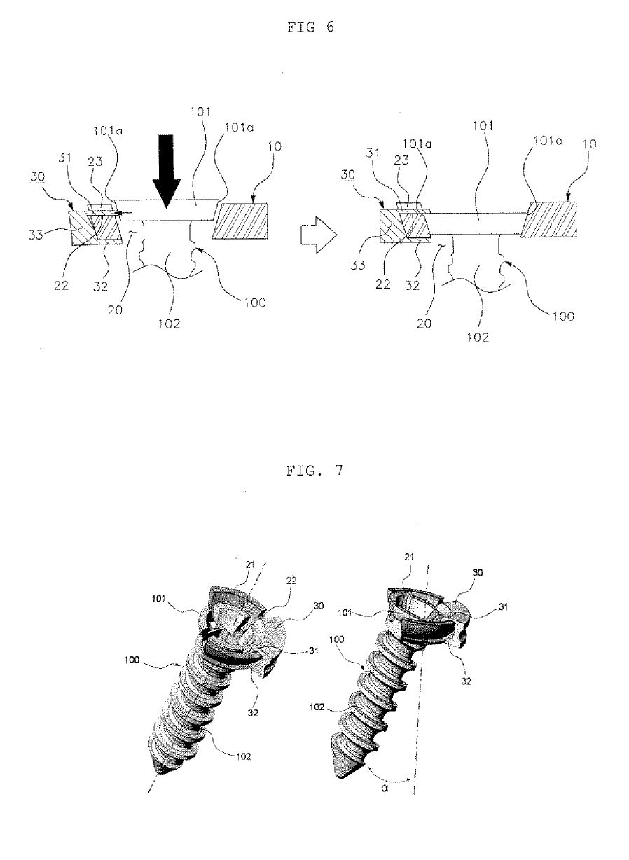

Accordingly, as shown in FIG. 5, the guide 30 can be pushed backwards in the direction of the center of the plate 10 or be returned to the direction of the center of the insertion holes 20, with the tension force of the arm 45.

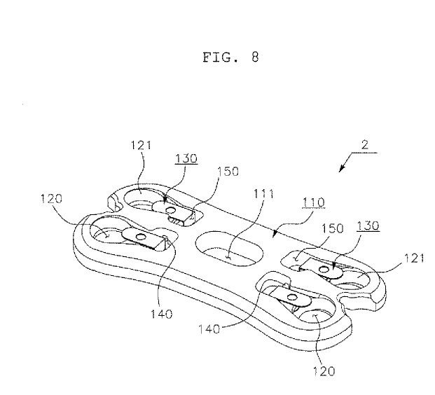

Hereinafter, the operation of an apparatus for fixing a cervical spine having a self tension part of the first exemplary embodiment of the present invention will be described in detail. FIGS. 6 to 7 are views showing an operating condition of an apparatus for fixing a cervical spine according to a first exemplary embodiment of the present invention.

First, as shown in the left drawing of FIG. 6, a screw 100 provided with a head 101 at the upper part and a screw body 102 at the lower part for fixing the plate 10 of an apparatus for fixing a cervical spine according to the first exemplary embodiment of the present invention, enters into the insertion holes 20 of the plate 10, and then, the guide 30 with an elastic force provided from the arm 45 of the tension part 40 is pushed backward toward the center of the plate 10 by the side surface 101a of the entering head 101 while the guide 30 contacts the head 101 of the screw 100 that enters downward. This state refers to an “Unlock” condition wherein the plate is not yet fixed with the screw 100.

However, when the screw 100 enters further into the insertion holes 20, the head 101 drops along a slope of the upper jaw 31 and then intrudes into a space between the upper jaw 31 and the lower jaw 32. At this time, the head is protruded frontward with the tension force of the arm 45 that has been pushed rearward to push the guide 30 toward the center of the insertion hole 20 while the contact between the head 101 of the screw 100 and the guide 30 is released.

Then, as shown in the right drawing of FIG. 6, the bottom surface 31 a of the upper jaw 31 that passes through the upper surface of the channel 22 and is protruded toward the center of the insertion holes 20, comes in contact with the upper surface of the head 101 of the screw 100, and the upper surface of the head 101 of the screw 100 is hooked over the bottom surface 31a of the upper jaw 31, thereby placing the screw 100 in “locking” state.

Under such a locking state of the screw 100, a phenomenon of screw retreatment of the apparatus for fixing a cervical spine can be prevented since the head 101 of the screw 100 is hooked over the upper jaw 31 of the guide 30.

FIG. 7 is a partial sectional view showing an apparatus for fixing a cervical spine according to a first exemplary embodiment of the present invention under such a locking condition, taken along a part of an inner wall 21 of an insertion hole 20.

As shown in the left drawing of FIG. 7, it is a matter of course that in the apparatus for fixing a cervical spine according to an embodiment of the present invention the screw 100 may enter into vertically the plate 10 to be locked, and further, as shown in the right drawing of FIG. 7, in the apparatus for fixing a cervical spine according to an embodiment of the present invention, when the screw 100 enters into the place 10 with a predetermined deviation angle (a) rather than a vertical direction, the guide 30 will rotate right/leftward in the engaged channel groove 22 along the deviation angle (a) of the head 101 of the screw 100 that enters therein, and the head 101 of the screw 100 will be hooked over the upper jaw 31 of the rotated guide 30.

Accordingly, according to the present invention, problems which may be caused by the conventional screw that is inserted at various angles during the operation of the screw for fixing a cervical spine can be solved at once, and moreover, the screw for the apparatus for fixing a cervical spine can be fixed simply and conveniently without paying attention to the entering angle of the screw to be fixed.

Next, a configuration and operation of the apparatus for fixing a cervical spine of the second exemplary embodiment of the present invention will be described.

The apparatus for fixing a cervical spine 2 of the second exemplary embodiment of the present invention is provided with the guide 30 and the tension part 40, which are modified from those in the first exemplary embodiment of the present invention, and hereinafter, the configuration and operation of the apparatus for fixing a cervical spine will be described in detail referring to FIGS. 8 toll.

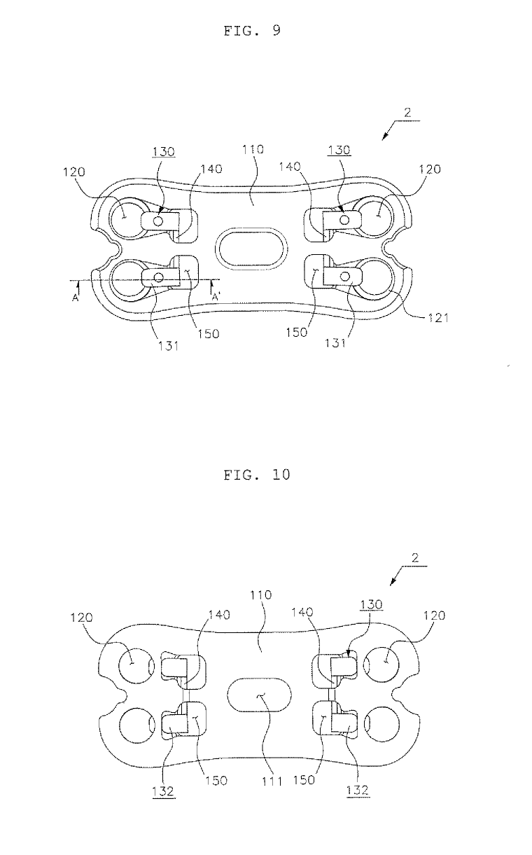

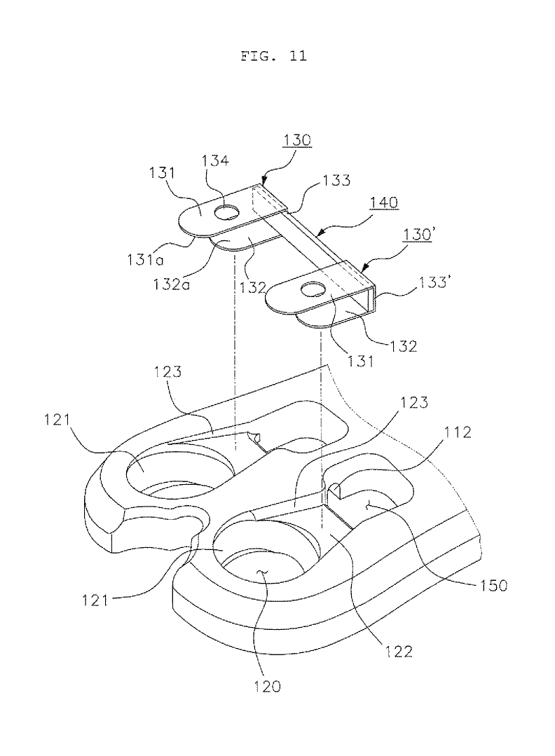

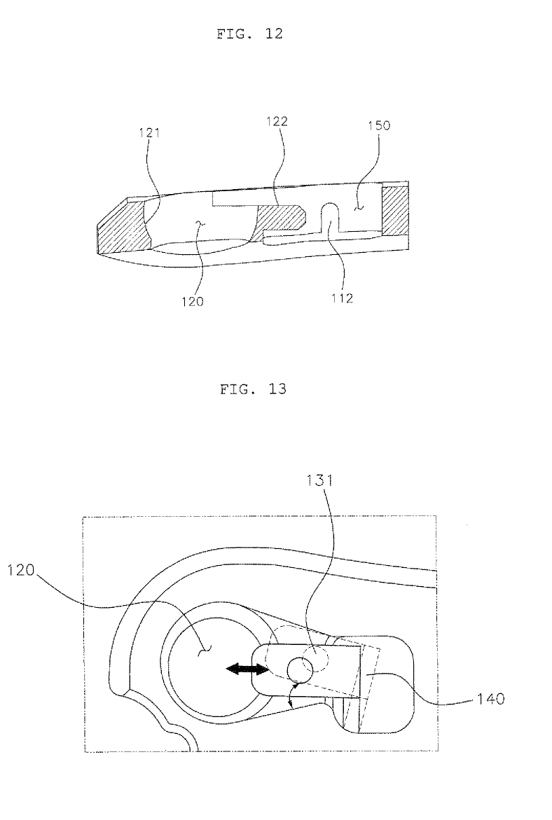

FIG. 8 is a perspective view showing an apparatus for fixing a cervical spine according to a second exemplary embodiment of the present invention, FIG. 9 is a plan view showing an apparatus for fixing a cervical spine according to a second exemplary embodiment of the present invention, FIG. 10 is a bottom view showing an apparatus for fixing a cervical spine according to a second exemplary embodiment of the present invention, FIG. 11 is an exploded perspective view showing an apparatus for fixing a cervical spine according to a second exemplary embodiment of the present invention, and FIG. 12 is a sectional view taken along line A-A of FIG. 9.

Referring to FIGS. 8 to 10, the apparatus for fixing a cervical spine 2 according to the second exemplary embodiment of the present invention is provided with a plate 110 of a flat shape, and the plate 110 may be made preferably of a rigid metal such as titanium, etc., similarly to the first exemplary embodiment of the present invention.

The plate 110 is shaped as a quadrangle, and each edge thereof is curved.

Further, inside of each edge of the plate 110, one or more insertion holes 120 into which screws 100 for fixing the plate 110 to the cervical spine are inserted are perforated through the plate 110 and similar to the first exemplary embodiment of the present invention, two, six, eight or more insertion holes may be provided depending on the fixation level of a cervical spine fixing operation.

Further, an inner wall 121 of the insertion hole 120 is curved to be meshed with the shape of a head 101 of a screw 100.

Additionally, an extension groove 150 is provided through the plate 110 between the insertion hole 120 and a through-hole 111 formed at the center of the plate 110, and a channel groove 122 is formed at the boundary of the insertion holes 120 and the extension groove 150, with stepped surfaces 123 each having a predetermined height from the surface of the plate 110 at both sides.

A guide 130 for locking the screw 100 that is inserted into the insertion holes 120 is inserted into the channel groove 122.

As shown in the exploded perspective view of FIG. 11, the guide 130 is formed integrally by connecting an upper jaw 131 and a lower jaw 132 in flat shapes, which are extended horizontally, with a connective surface 133 that is extended vertically.

Further, the tension part 140 with tension force in a plate or a rod shape is attached to the inside of the connective surface 133 of the guide 130 at one side and the connective surface 133′ of the guide 130′ at the other side to form a pair of two guides 130 as configured above.

At this time, as shown in FIG. 12, the tension part 140 is inserted into the insertion groove 112 formed at the lower part of the channel groove 122, and under this condition, the guides 130 and 130′ are connected to the channel groove 122, respectively such that the channel groove 122 is inserted between the bottom surface 131a of the upper jaw 131 and the upper surface 132a of the lower jaw 132 of the respective guides 130 and 130′.

Then, the upper jaw 131 of the connected guide 130 passes through the upper surface of the channel groove 122 and is protruded toward the center of the insertion holes 120, and the bottom surface 131a of the protruded upper jaw 131 contacts an upper surface of the head 101 of the screw 100 that is entering into the insertion hole 120, thereby locking the screw 100 and providing a hooking operation to the screw 100 not to retreat.

As shown in FIG. 12, the engaged guide 130 can rotate right/leftward until the upper jaw 131 and the lower jaw 132 are hooked over the stepped surfaces 123 at both sides of the channel groove 122, respectively.

The tension part 140 that is attached to the guide 130 has a self-tension force for pushing the guide 130 toward the center of the plate 110.

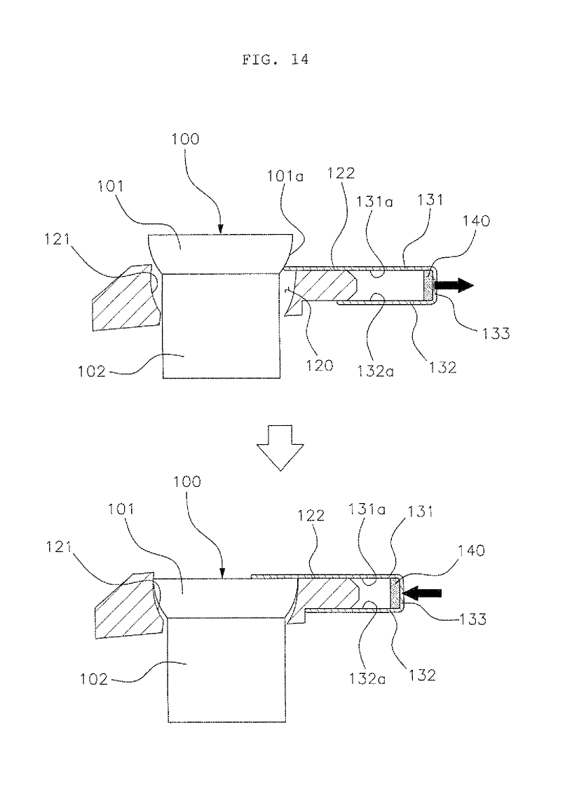

Accordingly, as shown in FIG. 13, the guide 130 rotates such that the guide is pushed toward the center of the plate 110 with the tension force of the tension part 140, and returns again toward the center of the insertion holes 120 with the tension force of the tension part 140.

Meanwhile, the number 134, which is not described, refers to a hole formed through the upper jaw 131 of the guide 130.

The apparatus for fixing a cervical spine 2 having a self tension part according to the second exemplary embodiment of the present invention performs same operation as the apparatus for fixing a cervical spine 1 according to the first exemplary embodiment of the present invention; the operation of the apparatus for fixing a cervical spine 2 according to the second exemplary embodiment of the present invention will be described referring to an operating condition of the screw being inserted in FIG. 14.

First, as shown in the upper drawing of FIG. 14, the screw 100 for fixing the plate 110 according to the second exemplary embodiment of the present invention enters into the insertion holes 120 wherein the screw 100 includes a head 101 at the upper part and a screw body 102 at the lower part.

Then, the guide 130 that is given with a resilience from the arm 45 of the tension part 40 is pushed backward toward the center of the plate 100 by the side surface 101a of the head 101 while the guide 130 contacts the head 101 of the screw 100 that enters downward. This condition refers to an “Unlock” state wherein the plate is not yet fixed with the screw 100.

However, when the screw 100 enters further into the insertion holes 120, the head 101 drops along a slope of the upper jaw 131 of the guide 130 and then intrudes into a space between the upper jaw 131 and the lower jaw 132.

At this time, the contact between the head 101 of the screw 100 that intrudes into a space between the upper jaw 131 and the low jaw 132 and the guide 130 is released and the guide 130 returns again toward the center of the insertion holes 120 with the tension force of the tension part 140 that has been pushed backward.

Then, as shown in the lower drawing of FIG. 14, the bottom surface 131a of the upper jaw 131 that passes through the upper surface of the channel 122 and is protruded toward the center of the insertion holes 120 comes in contact with the upper surface of the head 101 of the screw 100, and the upper surface of the head 101 of the screw 100 is hooked over the bottom surface 131a of the upper jaw 131 under this condition, thereby placing the screw 100 in a “locking” state.

Under such a locking condition of the screw 100, a phenomenon of screw retreatment of the apparatus for fixing a cervical spine can be prevented since the head 101 of the screw 100 is hooked over the upper jaw 131 of the guide 130.

Meanwhile, according to the apparatus for fixing a cervical spine 2 of the second exemplary embodiment as operated above, even when the screw 100 enters into the plate, with a predetermined deviation angle (a) rather than a vertical direction, the guide 130 can rotate right/leftward in the engaged channel groove 122 along the deviation angle (a) of the head 101 of the screw 100 that enters, and thus the screw for the apparatus for fixing a cervical spine can be fixed simply and conveniently without paying attention to the entering angle of the screw to be fixed at an operation by using the apparatus for fixing a cervical spine 1 according to the first exemplary embodiment of the present invention.

According to the apparatus for fixing a cervical spine of the present invention, the guide that rotates at a predetermined angle inside the insertion hole of the screw is fixed in close contact along the head of the screw that is entering at various angles, thereby fixing a screw firmly regardless of entering angle of the screw.

Additionally, according to the apparatus for fixing a cervical spine of the present invention, the operator can execute the operation by using the apparatus for fixing a cervical spine much simply and easily without paying attention to the entering angle of the screw to be fixed, thereby reducing the time for the operation.

While an apparatus for fixing a cervical spine of the present invention is described in detail referring to the accompanying drawings, however, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

1. An apparatus for fixing a cervical spine having a self tension part wherein a screw is inserted into one or more insertion holes, comprising:
an extension groove formed through a plate between the insertion holes and the center of the plate;
a channel groove formed with stepped surfaces at both sides at the boundary of the insertion holes and the extension groove;
a guide that is connected to the channel groove, rotates left/rightward in the channel groove and locks the screw to be inserted into the insertion holes; and
a tension part that gives a pushing force the guide to be pushed toward the center of the plate and allows the guide to be rotated in the channel groove.

2. The apparatus for fixing a cervical spine having a self tension part of claim 1, wherein the tension part comprises:
a base extending toward the center of the plate at one side of an edge of the extension groove;
a first curved part that is bent towards the guide at the end of the base;
a second curved part that is bent in parallel with the connective surface of the guide at the end of the first curved part; and
an arm formed with the contact surface between the second curved part and the connective surface of the guide.

3. The apparatus for fixing a cervical spine having a self tension part of claim 2, wherein the guide is formed integrally in a flat shape by connecting an upper jaw and a lower jaw, which are extended horizontally, with a connective surface that is extended vertically wherein the channel groove of the plate is connected between the bottom surface of the upper jaw and the upper surface of the lower jaw, and further the bottom surface of the upper jaw contacts an upper surface of the head of the screw, thereby locking the screw.

4. The apparatus for fixing a cervical spine having a self tension part of claim 1, wherein the guide is formed integrally in a flat shape by connecting an upper jaw and a lower jaw, which are extended horizontally, with a connective surface that is extended vertically wherein a tension part with a tension force is attached to the insides of the connective surface of the guide at one side and the connective surface of the guide at the other side wherein two guides form a pair, the tension part is inserted into the insertion groove formed at the lower part of the channel groove so that the channel groove is inserted between the bottom surface of the upper jaw and the upper surface of the lower jaw of the respective guides and the respective guides are connected to the channel groove, and the bottom surface of the upper jaw of the engaged guide contacts an upper surface of the head of the screw, thereby locking the screw.
